(19) United States
(12) Patent Application Publication
(10) Pub. No.: US 2024/0115798 A1
(43) Pub. Date: Apr. 11, 2024

(54) GRAVITY FLOW HYDRO-CLEANING SYSTEM

(71) Applicant: Maria ELENA HERNANDEZ MORA, Miami Springs, FL (US)

(72) Inventor: Maria ELENA HERNANDEZ MORA, Miami Springs, FL (US)

(21) Appl. No.: 17/962,445

(22) Filed: Oct. 7, 2022

(51) Int. Cl. A61M 3/02

(52) U.S. Cl. CPC A61M 3/0279; A61M 3/0254

(57) ABSTRACT

A disposable gravity flow hydro-cleaning system. The disposable gravity flow hydro-cleaning system comprises of an irrigation gun that has a pump that is attached to a tubing that is connected to an elevated solution dispenser The elevated solution dispenser contains a solution that is used to remove debridement from a wound of a patient. The solution dispenser is either a solution bag or a solution bottle.

### BACKGROUND

The present invention is directed to a gravity flow hydro-cleaning system that is used to remove debridement from a wound.

The invention uses a solution of chloride, sterile water, and/or a solution of super-oxidation. to remove debridement from a wound.

The invention is manually controlled by a physician and does not require and electronics to function. It operated based on gravity and a manual pump extraction system.

Presently, there are electro-mechanical wound cleaning systems that use electric pumps to clean wounds, the systems are expensive and lend themselves to contamination, for the systems must be re-used when treating patients. If a careless practitioner uses the existing electro-mechanical wound cleaning systems, a cross contaminations may occur in the patient treatment rooms, clinics, or hospitals.

The present invention is designed to be disposable after the treatment of a patient.

The relative low cost to produce and sell the present invention discourages the need of the invention having to be re-used from patient to patient.

Presently, debridement is removed from wounds of patients using devices that are made of latex, syringes, and electro-mechanical sprayers that over irrigate a wound. The devices lend themselves to contamination. Contamination occurs when wounds are over irrigated and the over irrigation causes surrounding contaminants to inadvertently seep into the wound.

The present invention eliminates the need to use electro-mechanical wound cleaning systems to clean the debridement from a wound, and thereby reduces the chances of contamination occurring in a patient treatment facility.

### SUMMARY

The present invention is a disposable gravity flow hydro-cleaning system. The disposable gravity flow hydro-cleaning system comprises of an irrigation gun that has a pump that is attached to a tubing that is connected to an elevated solution bag. The elevated solution bag contains a solution that is used to remove debridement from a wound of a patient.

An object of the present invention is to provide a disposable gravity flow hydro-cleaning system that can be used to remove debridement from a wound of a patient.

Another object of the present invention is to provide a cost and effective gravity flow hydro-cleaning system that can be used to remove debridement from a wound of a patient.

Yet another object of the present invention is to provide a disposable gravity flow hydro-cleaning system. that will prevent cross infection in a patient treating facility.

Still another object of the present invention is to provide a gravity disposable flow hydro-cleaning system that eliminates the need of using an electro-mechanical device to remove debridement from a wound of a patient.

### BRIEF DESCRIPTION OF THE DRAWINGS

These and other features, aspects, and advantages of the present invention will become better understood with regards to the following description, appended claims, and drawings where:

FIG. 1 shows a side view of the present invention;

FIG. 2 shows a perspective view of the present invention;

FIG. 3 shows an exploded perspective view of the present invention; and

FIG. 4 shows a perspective view of the present invention hooked up to a solution bag.

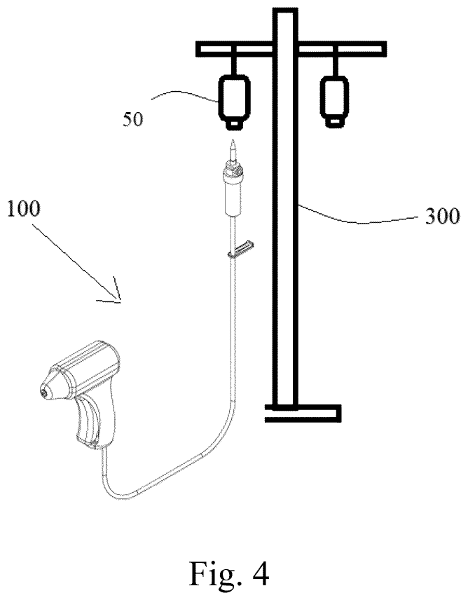

### DESCRIPTION

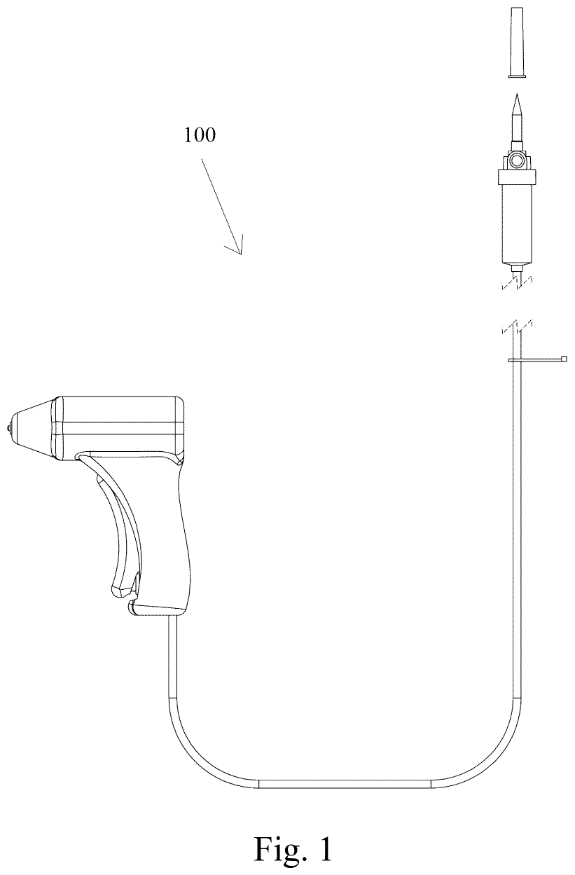

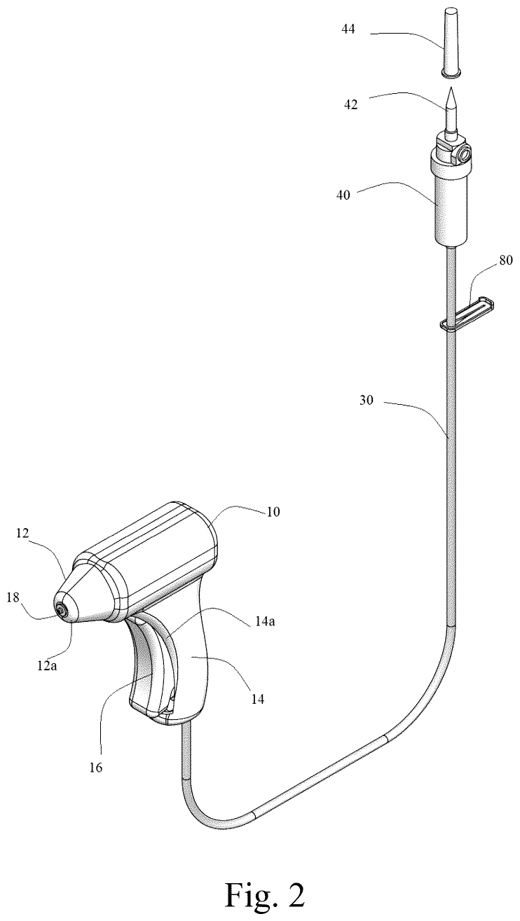

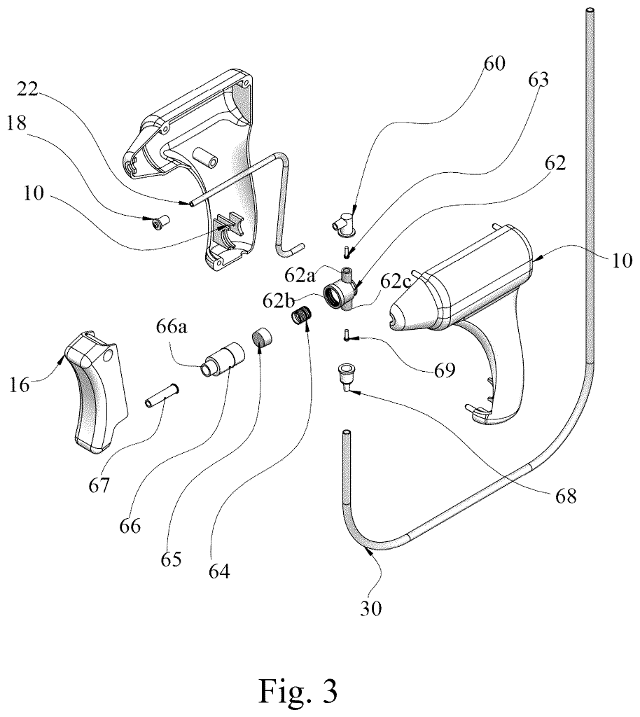

As seen in FIGS. 1-3, the present invention is a gravity flow hydro-cleaning system 100 that is used to remove debridement from a wound of a patient. The gravity flow hydro-cleaning system 100 comprises an irrigation gun 10, the irrigation gun 10 has a barrel 12 and a handle 14. The irrigation gun 10 comprises a trigger 16 that is positioned on a front section 14a of the handle 14 that is configured to pump a miniature pump 20, a nozzle 18 that is positioned on a front section 12a of the barrel 12, the miniature pump 20 that is housed in the irrigation gun 10 and that is operatively connected to the trigger 16, and a nozzle to pump tubing 22, the nozzle to pump tubing connects 22 the nozzle 18 to the miniature pump 20. A sterile tubing 30 that is connected to the miniature pump 20. A spike and drip chamber assembly 40 that is connected to the sterile tubing 30. And, a solution dispenser 50 that holds a solution that is used to remove debridement from a wound of a patient. The solution dispenser 50 is configured to be secured to either an IV Pole 300 or to a raised structure. The solution dispenser 50 is connected to the spike and drip chamber assembly 40.

In preferred embodiments, the solution dispenser 50 is either a solution bag or a solution bottle.

In preferred embodiments of the present invention, the miniature pump 20 comprises of a ninety degree tube fitting 60 that attaches to a top section 62a of a one way valve 62 after a first valve plunger 63 is placed in between the ninety degree tube fitting 60 and the top section 62a of the one way valve 60, a spring 64 that inserts within a front section 62b of the one way valve 62, a pump cylinder 65 that is placed over the spring 64, a pump housing 66 that attaches to the front section 62b of the one way valve 60, a trigger shaft 67 that inserts within a front section 66a of the pump housing 66 and that attaches to the trigger 16, and a tube fitting 68 that attaches to a lower section 60c of the one way valve 60 after a second valve plunger 69 is placed between the tube fitting 68 and the lower section 60c of the one way valve 60, the ninety degree tube fitting 60 connects to the nozzle and pump tubing 22, and the tube fitting 68 connects to the sterile tubing 30.

In preferred embodiments, the gravity flow hydro-cleaning system 100 comprises a slide clamp 80 that is configured to be placed over the sterile tubing 30 when the flow of the solution is to be cut off and a spike cover 44 that is configured to be placed of a spike 42 of the spike and drip chamber assembly 40.

An advantage of the present invention is that it provides a disposable gravity flow hydro-cleaning system that is used to remove debridement from a wound of a patient.

Another advantage of the present invention is that it provides a cost and effective gravity flow hydro-cleaning system that is used to remove debridement from a wound of a patient.

Yet another advantage of the present invention is that it provides a disposable gravity flow hydro-cleaning system that prevents cross infection in a patient treating facility.

Still another advantage of the present invention is that it provides a gravity disposable flow hydro-cleaning system that eliminates the need of using an electro-mechanical device to remove debridement from a wound of a patient.

While the inventor's description contains many specificities, these should not be construed as limitations of the gravity flow hydro-cleaning system, but rather as an exemplification of several preferred embodiments thereof, any other variations may be possible. Accordingly, the scope should be determined not by the embodiments illustrated, but by the specification, the drawings, and the claims and any legal equivalent thereof.

What is claimed is:

1. A gravity flow hydro-cleaning system that is used to remove debridement from a wound of a patient, the gravity flow hydro-cleaning system comprises:
an irrigation gun, the irrigation gun has a barrel and a handle, the irrigation gun comprises:
a trigger that is positioned on a front section of the handle that is configured to pump a miniature pump,
a nozzle that is positioned on a front section of the barrel,
the miniature pump that is housed in the irrigation gun and that is operatively connected to the trigger, and
a nozzle to pump tubing, the nozzle to pump tubing connects the nozzle to the miniature pump;
a sterile tubing that is connected to the miniature pump;
a spike and drip chamber assembly that is connected to the sterile tubing; and
a solution dispenser that holds a solution that is used to remove debridement from a wound of a patient, the solution dispenser is configured to be secured to either an IV Pole or to a raised structure, the solution dispenser is connected to the spike and drip chamber assembly.

2. The gravity flow hydro-cleaning system that is used to remove debridement from a wound a patient of claim 1, wherein the miniature pump comprises of a ninety degree tube fitting that attaches to a top section of a one way valve after a first valve plunger is placed in between the ninety degree tube fitting and the top section of the one way valve, a spring that inserts within a front section of the one way valve, a pump cylinder that is placed over the spring, a pump housing that, attaches to the front section of the one way valve, a trigger shaft that inserts within a front section of the pump housing and that attaches to the trigger, and a tube fitting that attaches to a lower section of the one way valve after a second valve plunger is placed between the tube fitting and the lower section of the one way valve, the ninety degree tube fitting connects to the nozzle and pump tubing, and the tube fitting connects to the sterile tubing.

3. The gravity flow hydro-cleaning system that is used to remove debridement from a wound a patient of claim 1, the gravity flow hydro-cleaning system comprises a slide clamp that is configured to be placed over the sterile tubing when the flow of the solution is to be cut off and a spike cover that is configured to be placed of a spike of the spike and drip chamber assembly.

4. The gravity flow hydro-cleaning system that is used to remove debridement from a wound a patient of claim 1, wherein the solution dispenser is a solution bag.

5. The gravity flow hydro-cleaning system that is used to remove debridement from a wound a patient of claim 1, wherein the solution dispenser is a bottle.
